(19) United States
(12) Patent Application Publication
(10) Pub. No.: US 2011/0028856 A1
(43) Pub. Date: Feb. 3, 2011

(54) System for Heart Performance Characterization and Abnormality Detection

(75) Inventor: Hongxuan Zhang, Palatine, IL (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS USA, INC., Malvern, PA (US)

(21) Appl. No.: 12/749,546

(22) Filed: Mar. 30, 2010

Related U.S. Application Data
(60) Provisional application No. 61/229,317, filed on Jul. 29, 2009.

(51) Int. Cl. A61B 5/0402

(52) U.S. Cl. 600/515

(57) ABSTRACT

A system improves analysis, diagnosis and characterization of cardiac function signals (including surface ECG signals and intra-cardiac electrograms) based on cardiac electrophysiological activity momentum computation, characterization and mapping. The system calculates an electrophysiological signal momentum of different portions of cardiac signals including a timing, location and severity of cardiac pathology and improves reliability of diagnosis, detection, mapping to an identified medical condition, and characterization. The system improves identification of cardiac disorders, differentiation of cardiac arrhythmias, characterization of pathological severity, prediction of life-threatening events and supports evaluation of drug administration effects.

This is a non-provisional application of provisional application Ser. No. 61/229,317 filed Jul. 29, 2009, by H. Zhang.

### FIELD OF THE INVENTION

This invention concerns a system for heart performance characterization and abnormality detection by determining rate of change of amplitude of an electrical signal representing heart electrical activity over at least a portion of a heart beat cycle.

### BACKGROUND OF THE INVENTION

Different portions of cardiac electrophysiological signals are associated with activities and functions of different cardiac tissue and circulation systems. Usually, surface ECG (including intra-cardiac electrogram) signal analysis based on electrophysiological activity and time domain parameters of waveforms is used to detect cardiac arrhythmia. This is performed by detecting P wave disorders for atrial fibrillation (AF) and ST segment changes for myocardial ischemia and infarction, for example. However, known systems for cardiac arrhythmia identification and analysis based on ECG signals are typically subjective and need extensive expertise and clinical experience for accurate interpretation and appropriate cardiac rhythm management.

Early arrhythmia recognition and characterization, such as of myocardial ischemia and infarction, is desirable to manage rhythm associated with cardiac disorders and irregularities. Known methods use waveform morphology and time domain parameter analysis of depolarization and repolarization functions involving P wave, QRS complex, ST segment and T wave analysis for cardiac arrhythmia monitoring and identification. However, such known methods are subjective and time-consuming, and require expertise and clinical experience for accurate interpretation and proper cardiac rhythm management. Known systems typically fail to provide adequate information concerning cardiac electrophysiological function interpretation for tissue impairment and arrhythmia localization. Also known diagnostic methods typically focus on time characteristics (such as peak amplitude, latency) or frequency characteristics (power, spectrum) domain changes and analysis, which may not accurately capture small signal changes in a signal portion (such as of a P wave, QRS complex, ST segment, for example). Consequently, known methods may have a high failure rate for arrhythmia detection and have a substantial false alarm detection rate.

Known cardiac diagnostic methods based on amplitude (voltage) changes and variation may be inadequate for cardiac function evaluation and pathology diagnosis. Known power spectrum and frequency analysis methods may not be able to map signal frequency variation to cardiac pathological functional changes. Known systems fail to comprehensively capture and diagnose QRS complex signal portions for myocardial ischemia analysis and fail to qualitatively and quantitatively characterize changes and predict pathological trends including a real time increasing trend of a cardiac arrhythmia, such as a pathology trend from low risk to medium, and then to high risk (severe and fatal) rhythm, for example. Known clinical methods for cardiac arrhythmia calculation and evaluation may generate inaccurate and unreliable data and results because of unwanted noise and artifacts. Environmental noise and patient movement artifacts, such as electrical interference, can distort a waveform and make it difficult to detect R wave and ST segment elevation accurately. A system according to invention principles addresses these deficiencies and related problems.

### SUMMARY OF THE INVENTION

A system calculates an electrophysiological signal momentum value for different portions of a cardiac signal for use in determining cardiac cycle timing, location and severity of cardiac pathology and maps calculated values to an identified medical condition. A system for heart performance characterization and abnormality detection includes an interface for receiving an electrical signal indicating electrical activity of a patient heart over at least one heart beat cycle. A signal processor calculates a signal characteristic value comprising a summation of values of rate of change of amplitude of the electrical signal over at least a portion of a heart beat cycle. A comparator compares the calculated signal characteristic value with a threshold value to provide a comparison indicator. A patient monitor, in response to the comparison indicator indicating the calculated signal characteristic value exceeds the threshold value, generates an alert message associated with the threshold.

### BRIEF DESCRIPTION OF THE DRAWING

FIG. 1 shows a system for heart performance characterization and abnormality detection, according to invention principles.

FIG. 2 illustrates excitation force in cardiac tissue and signal momentum calculation, according to invention principles.

FIG. 3 shows an equation for signal momentum calculation, according to invention principles.

FIG. 4 shows a flowchart of a process for signal momentum analysis based measurement, monitoring, calculation and characterization, according to invention principles.

FIG. 5 illustrates momentum calculation and diagnosis of myocardial ischemia, according to invention principles.

FIG. 6 illustrates intra-cardiac catheter EP signal based momentum analysis involving cardiac internal excitation force mapping of a heart chamber, muscle, according to invention principles.

FIG. 7 shows a flowchart of a process used by a system for heart performance characterization and abnormality detection, according to invention principles.

### DETAILED DESCRIPTION OF THE INVENTION

A system improves analysis, diagnosis and characterization of cardiac function signals (including surface ECG signals and intra-cardiac electrograms) based on cardiac electrophysiological activity momentum computation. The system calculates an electrophysiological signal momentum value for different portions of cardiac signals. The value is used for determining timing, location and severity of cardiac pathology and improves reliability of diagnosis, detection, mapping to an identified medical condition, and characterization. The system improves identification of cardiac disorders, differentiation of cardiac arrhythmias, characterization of pathological severity, prediction of life-threatening events and supports evaluation of drug administration effects.

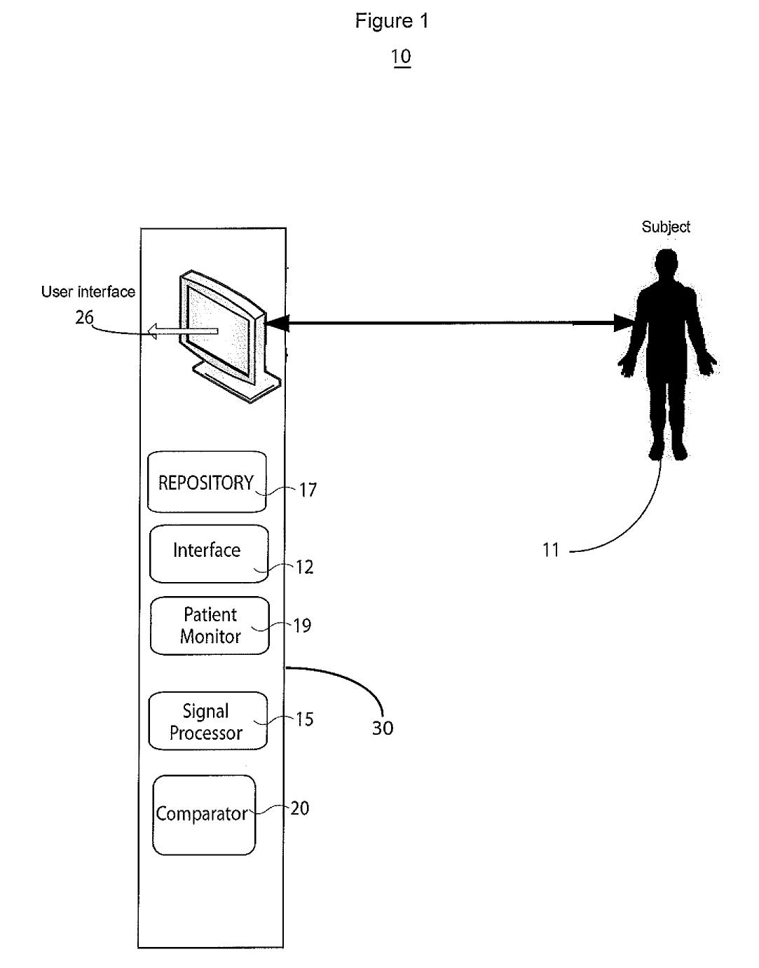

FIG. 1 shows system 10 for heart performance characterization and abnormality detection. System 10 analyzes electrophysiological signals (including surface ECG, intra-cardiac electrograms, and heart activity signals, such as cardiac sound waveform) by deriving a signal momentum based cardiac function diagnosis value. System 10 employs cardiac signal segmentation and identifies cardiac pathology based on electrophysiological signal momentum and variation analysis. System 10 further provides catheter multi-channel signal and tissue site electrophysiological signal momentum analysis using heart and circulation function mapping (local and global) involving 2D or 3D cardiac momentum mapping. System 10 determines signal activity momentum values for P wave, QRS complex and ST segments, for example, as an objective, sensitive, accurate and reliable assessment. The signal activity momentum values are used to provide detailed information indicating severity of pathology, location of an abnormal function and tissue (muscle, chamber) and a time within a heart beat cycle showing a problem.

The system electrophysiological signal momentum determination involves different signal portion momentum calculations, such as for P wave versus QRS complex and QRS complex versus ST segment. System 10 characterizes signal distortion and cardiac functional abnormality along a signal pathway with time synchronization to identify abnormality in pacing force, pacing excitation conduction and variation in a cardiac chamber, tissue and circulation pathway. The electrophysiological signal momentum determination identifies acute changes and abnormality within cardiac signals and electrophysiological activities by detection of acute AF and acute ischemia events. The signal momentum (signal dynamic analysis) is also advantageously usable for other kinds of signal processing involving biological force or oximetric signals such as hemodynamic pressure signals, SPO2 blood oxygen saturation and blood flow signals

When certain abnormality or clinical events occur, usually cardiac tissue is affected first and a pacing excitation conduction process is impacted and shows abnormal variation. The electrophysiological signal momentum analysis and calculation identifies pacing and excitation force and associated time duration, to detect cardiac arrhythmias, characterize pathological severity, predict life-threatening events, and evaluate drug delivery effects.

System 10 comprises at least one computer system, workstation, server or other processing device 30 including interface 12, repository 17, patient monitor 19, signal processor 15, comparator 20 and a user interface 26. Interface 12 receives an electrical signal indicating electrical activity of a patient 11 heart over at least one heart beat cycle. Signal processor 15 calculates a signal characteristic value comprising a summation of values of rate of change of amplitude of the electrical signal over at least a portion of a heart beat cycle. Comparator 20 compares the calculated signal characteristic value with a threshold value to provide a comparison indicator. Patient monitor 19, in response to the comparison indicator indicating the calculated signal characteristic value exceeds the threshold value, generates an alert message associated with the threshold.

The electrical pathways in the heart connect one part to another such as the S-A node to the A-V node, for instance. This heart conduction of electrical signals for pacing and excitation along the pathways cause the heart to contract (or beat) and relax. Heart cycle stages include a first step, the S-A node (natural pacemaker) creates an electrical signal. In a second step, the electrical signal follows natural electrical pathways through both atria causing the atria to contract, which helps push blood into the ventricles. In a third step, the electrical signal reaches the A-V node (electrical bridge) and the signal pauses to give the ventricles time to fill with blood. In a fourth step, the electrical signal spreads through the His-Purkinje system causing the ventricles to contract and push blood out to lungs and body.

Physics and dynamics principles indicate force changes the status of a system. Cardiac internal myocardial stimulation and excitation forces cause transition from a cardiac rest stage to a cardiac depolarization and repolarization stage. Different portions of cardiac signals (such as ECG and ICEG signals) reflect an excitation force and muscle response. The muscle electrophysiological response is used by system 10 to extract data indicating the force and its variation. By considering the mass of the muscle which corresponds to different portions of heart tissue and signal response, cardiac signal momentum is used to track cardiac excitation force, and characterize pathology and events via momentum distortion, deviation and changes.

Excitation force:

$$F = ma = m \cdot \frac{d V}{d t} = m \cdot \frac{{d}^{2} A}{{d}^{2} t} \qquad equation 1$$

FIG. 3 shows equation 2 for signal momentum calculation.

(Signal momentum:

$$P = {\sum }_{ROI} F \cdot Δ t = {\sum }_{ROI} m \cdot Δ v = {\sum }_{ROI} m \cdot Δ ( \frac{A}{t} ) ) .$$

Where A is signal Amplitude.

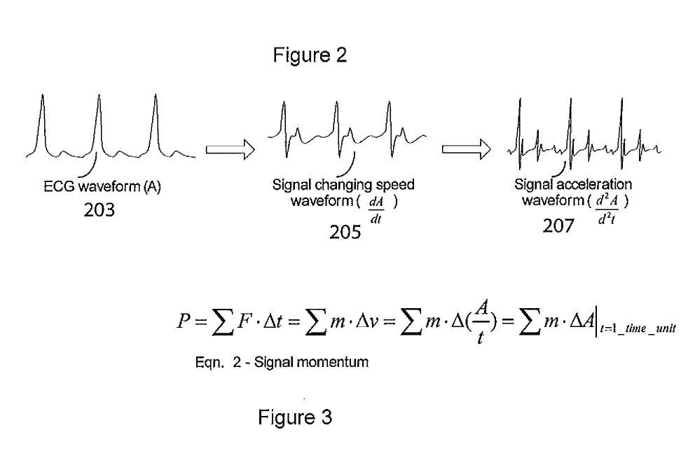

FIG. 2 illustrates excitation force in cardiac tissue and signal momentum calculation. Specifically, FIG. 2 shows an ECG waveform (A) 203, velocity of the ECG waveform

$$( \frac{d A}{d t} )$$

205 and acceleration of the ECG waveform

$$( \frac{{d}^{2} A}{{d}^{2} t} )$$

207. Excitation force changes the electrophysiological signal and response of the heart and myocardial tissue. However, force measurement and characterization, especially for internal tissue excitation, is difficult and may be invasive. The system advantageously employs signal momentum determination to track and characterize the excitation force and heart (health) status changes. (Note in equation 2 of FIG. 3 t=1_time_unit means the incremental computation time step for momentum calculation and analysis.). In order to achieve non-invasive (or less invasive) monitoring, recording and diagnosis of cardiac tissue functions, system 10 (FIG. 1) uses signal momentum to analyze the excitation force deviation and force distribution of a ROI (region of interest), cardiac tissues, and signal pathways. By comparing the same tissue or electrophysiological response of different time portions, mass is the same and is treated as a constant parameter in the calculation and diagnosis procedures.

Signal momentum is,

$$P = {\sum }_{ROI} m \cdot v ,$$

In which, v is derived from the electrophysiological signals as described in FIG. 1 and is

$$\frac{d A}{d t} .$$

Deviation of the signal momentum is,

E(P),STD(P)(δ(P)),Δ(P)%

The signal momentum calculation performed by system 10 is not limited to calculation using

$$P = {\sum }_{ROI} m \cdot v ,$$

The signal momentum rate may also be calculated as,

$$Δ P = {\sum }_{ROI} m \cdot Δ v$$

in which, Δv may be determined as

$$\frac{{d}^{2} A}{{d}^{2} t} .$$

Signal momentum calculation provides a calculated parameter and index value from current electrophysiological signals, such as surface ECG and intra-cardiac electrogram signals. The signal momentum values capture the changing rate and mode of cardiac excitation force and corresponding cardiac function variation. Hence the signal momentum calculation data may be used for data mining and remodeling to extract mode by using linear or nonlinear mode recognition and modeling methods, such as AR modeling (Autoregressive model), ARMA modeling (Autoregressive integrated moving average)), Chaos modeling, Fuzzy modeling or artificial neuron network (ANN) modeling, for example. AR modeling is used as an alternative to use of a discrete Fourier transform (DFT) in the calculation of a power spectrum density function of a time series. The power spectrum gives information about the frequency content of a time series. In biomedical applications, AR modeling is used in spectral analysis of heart rate variability and electroencephalogram recordings. AR modeling provides a smoother and more easily interpretable power spectrum than DFT. For simplification of the pattern recognition and remodeling, system 10 performs AR modeling based signal momentum mode analysis involving data acquisition and filtering (electrophysiological signals) in a first step. In a second step, system 10 calculates signal momentum and momentum rate in which signal momentum data series are derived. System 10 in a third step uses AR model based pattern recognition and remodeling by calculating an AR spectrum and finding a good set of AR model coefficients aᵢ, (See Appendix). In a fourth step, system 10 performs signal momentum analysis, momentum deviation analysis, and momentum mode and pattern analysis.

System 10 in different embodiments employs different methods of AR modeling based signal momentum mode and pattern analysis. Mode comparison is used to track difference between signal momentum values of a normal (healthy) electrophysiological (baseline) signal and current signals:

$${Momentum}_{AR\_mode} ( \% ) = \frac{{\sum }_{AR\_modeling} {( {a}_{1 i} - {a}_{2 i} )}^{2}}{{\sum }_{AR\_modeling} {( {a}_{1 i} )}^{2}}$$

In which, a₁ᵢ and a₂ᵢ stand for AR modeled spectral (coefficients) of Normal (healthy) and current (real time) data series. By using Momentum_{AR}_{_{—}}_{mode} (%) the momentum mode and pattern change and variation are captured and characterized. The momentum based calculation and analysis (including Signal momentum analysis, momentum deviation analysis, and momentum mode/pattern analysis) performed by system 10 provides early detection of pathology and clinical events, accurate characterization of the arrhythmia severity (especially of index value and deviation degree), and prediction of potential life risky events with drug delivery information. The momentum based signal interpretation may be performed on a windowed electrophysiological signal or a signal portion comprising multiple heart cycles. The momentum analysis can also be used for signal portion tracking and characterization, such as of a P wave portion for Atrial Fibrillation and an ST portion for myocardial ischemia or infarction.

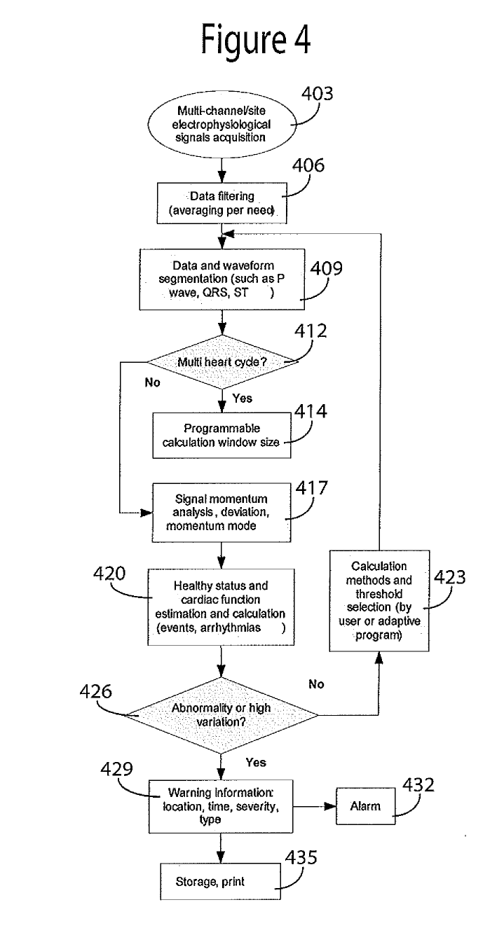

FIG. 4 shows a flowchart of a process for signal momentum analysis based measurement, monitoring, calculation and characterization performed by system 10 (FIG. 1). Interface 12 in step 403 acquires electrophysiological signals from multiple channels of a multi-channel intra-cardiac (e.g., basket) catheter indicating electrical activity at multiple cardiac tissue sites. Signal processor 15 in step 406 filters the acquired electrophysiological signals using a filter adaptively selected in response to data indicating clinical application (e.g. ischemia detection, rhythm analysis application). In step 409 processor 15 identifies different segments (QRS, ST, P wave segments, for example) of the filtered electrophysiological signals. In step 417, signal processor 15 calculates a signal momentum value, a deviation of a current signal momentum value from a previously determined momentum signal value and a momentum mode value, in response to a determination momentum is being calculated for a single heart cycle in step 412. Signal processor 15 calculates momentum values for electrophysiological signals from multiple channels of a multi-channel intra-cardiac (e.g., basket) catheter for analysis of the different channel signal momentum (such as to determine momentum pattern, mode and deviation). Processor 15 also determines the location, timing, severity and type of cardiac pathology and associated disease. Further, in response to a determination from predetermined calculation configuration data, that signal momentum is being calculated over multiple heart cycles in step 412, signal processor 15 adjusts momentum calculation to be performed over a particular window portion of multiple different heart cycles.

In step 420 signal processor 15 employs mapping information, associating ranges of a calculated momentum value or values derived from the momentum value, with corresponding medical conditions (e.g., arrhythmias) in determining patient medical conditions, events and patient health status. If signal processor 15 and comparator 20 in step 426 determines a medical condition indicating cardiac impairment or another abnormality is identified, patient monitor 19 in step 429 generates an alert message identifying the medical condition and abnormality and communicates the message to a user in step 432 and stores or prints the message and records the identified condition in step 435.

If signal processor 15 and comparator 20 in step 426 does not identify any medical condition potentially indicating cardiac impairment, signal processor 15 in step 423 iteratively repeats the process from step 409 using adaptively adjusted momentum computation parameters and comparison thresholds, time between ECG samples used, computation window size (i.e., portion of a heart cycle over which a momentum calculation is performed). System 10 uses the calculated signal momentum value to continuously monitor and quantify cardiac excitation force variation and distortion to achieve early detection of clinical events.

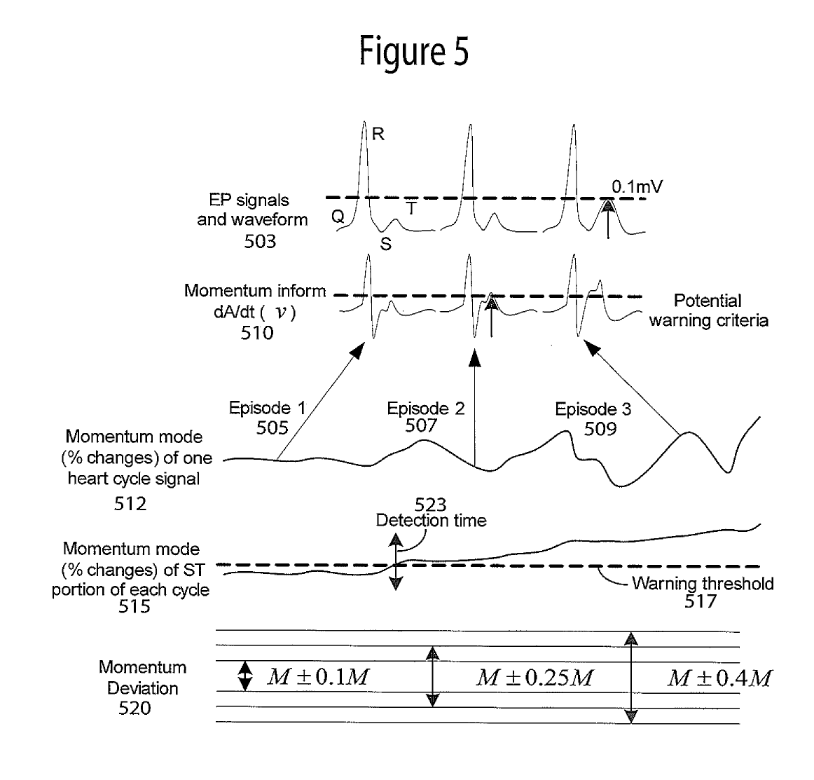

FIG. 5 illustrates momentum calculation and diagnosis of myocardial ischemia. FIG. 5 shows different stages, from healthy (Episode 1 505) to intermediate (Episode 2 507), to early ischemia (Episode 3 509). If relying on an EP signal amplitude (voltage) change, such as an ST segment level change (0.1 mV threshold), a user may need 20-30 minutes before an ST segment detection generates a warning. In contrast, signal processor 15 performs signal momentum value calculation and analysis to provide early analysis and detection using an index enabling a user to track myocardial function and perfusion procedure deviation indicating cardiac excitation force variation or myocardial muscle pathologies. Momentum information (taking mass as a constant) provides improved sensitivity and reliability and facilitates early detection of ischemia events. Processor 15 uses statistical analysis in adaptively dynamically adjusting a momentum value comparison threshold for clinical event detection. The signal momentum analysis is not limited to single heart cycle signal, and in one embodiment is performed for different portions of a cardiac cycle, such as depolarization (QRS), repolarization (ST, T wave). Further the momentum calculation is advantageously used for comparison of different heart chambers and to build a multi-channel model of cardiac function.

FIG. 5 illustrates different methods of myocardial ischemia detection. Waveform 503 shows the standard method involving detection of an ST segment portion of an electrophysiological signal exceeding a 0.1 mV threshold, for example. Waveform 512 shows a plot of momentum mode value of one heart cycle signal plotted against time together with corresponding momentum value waveform 510. Waveform 515 shows a momentum mode value of an ST segment portion of a heart and warning threshold 517. The momentum deviation ranges and thresholds are illustrated as adaptively increasing ranges 520 (±0.1M, ±0.25M, ±0.4M). Signal processor 15 performs momentum value and mode computation to identify acute changes in cardiac signals and heart function. Typically a physician uses an ST segment to track the changes and identify an acute ischemia event by calculating the ST segment magnitude (warning threshold is 0.1 mV). However, it is difficult to track and determine variation of an ST segment before it reaches the threshold (0.1 mV which is a typical standard threshold for clinical users). Furthermore, an acute ischemic event may occur a substantial time before the ST segment presents a significant elevation. For example, in FIG. 3, electrograms in episode 2 (507) indicate small changes and variation which are quantitatively captured and characterized. System 10 (FIG. 1) uses momentum analysis to precisely quantify ST segment changes including a variation and trend.

In one embodiment, an ST segment momentum of a healthy heart beat (baseline) has a value of 1 (normalized). Momentum mode calculation index value increases because of myocardial ischemia. In comparing the three episodes 505, 507 and 509 of cardiac monitoring, the momentum index value of episode 1 is 1.01, while momentum index values of episode 2 and 3 are 1.26 and 1.57 respectively. Once the value reaches 10% above baseline value (a threshold set for analysis in this example), an ischemia event warning is generated at detection time 523.

Compared with traditional ischemia event analysis based on ST segment magnitude, momentum calculation and mode analysis ischemia event detection may be significantly earlier and more reliable. System 10 adaptively adjusts detection threshold for cardiac arrhythmia quantification and selects a 10%, 25%, 40% level above baseline value as a threshold, for example. The detection threshold stability and reliability is adjusted by system 10 in response to analyzing statistical data including detection rate and probability of detection both for the patient concerned and for a population of patients having comparable demographic characteristics (age, weight, height, gender, pregnancy status) of the patient concerned. System 10 performs momentum analysis for a whole heart cycle and ROI (interesting portion in the heart beat cycle, such an ST segment or repolarization portion for cardiac ischemia event analysis). In one embodiment system 10 performs momentum mode analysis by advantageously averaging cardiac signals to reduce noise effects and increase signal to noise ratio.

In operation, signal processor 15 selects a baseline value as a value derived from the patient normal heart signal or a baseline value derived from a population of patients sharing comparable demographic characteristics with the patient concerned and momentum value index is unified as 1. Processor 15 performs signal segmentation to select a ROI portion such as an ST segment portion for ischemia detection. Processor 15 calculates a momentum value for this portion as the momentum index value

$${\sum }_{ST\_segment} m \cdot \frac{d A}{d t}$$

where m is the same for the same patient and can be unified as 1.

A user is also able to initiate an AR model based momentum index calculation. Signal processor 15 continuously calculates momentum values for episodes 505, 507 and 509, comprising normal, intermediate and early ischemia episodes. Processor 15 compares momentum index values of these episodes with corresponding baseline momentum values to provide normalized values 1.01 1.26 1.57. In one embodiment, processor 15 adaptively adjusts a detection threshold for initiating generation of a cardiac event warning in response to signal to noise ratio. For example, in response to determining a signal to noise ratio of 10:1, processor 15 selects a threshold of greater than 15% above baseline value, and selects a threshold of greater than 30% in response to determining a signal to noise ratio of 5:1 or less. Processor 15 adaptively selects a threshold in response to sensitivity and stability of arrhythmia detection and quantification. Usually a statistical analysis (such as a T test which assesses whether the means of two groups are statistically different from each other) is utilzed to get 95% confidence for detecting clinical events.

System 10 performs multi-channel and cardiac site electrophysiological signal momentum analysis for each site and maps momentum values to cardiac condition localized to particular cardiac sites. Signal momentum analysis including pattern, mode and deviation calculation facilitates tracking and characterization of cardiac electrophysiological signal distortion and variation. Furthermore, system 10 calculates cardiac excitation force along a heart muscle and pathway.

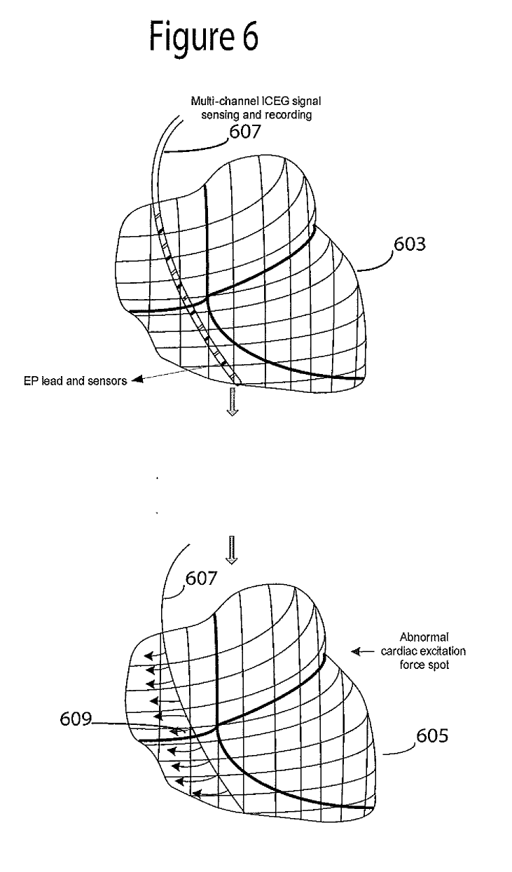

FIG. 6 illustrates intra-cardiac catheter EP signal based momentum analysis involving cardiac internal excitation force value mapping to an indicator of heart chamber muscle condition and muscle and signal pathway condition. Diagram 603 shows multi-channel ICEG catheter 607 concurrently sensing electrophysiological (EP) signals from multiple sites within a heart for recording by system 10. Signal processor 15 calculates momentum value, mode value and deviation value of the sensed EP signals and uses the values to analyze heart excitation force change and variation along catheter 607. Processor 15 maps detected momentum parameters and their change to one or more abnormal tissue sites and detects arrhythmias to facilitate prevention of life threatening events. Diagram 605 illustrates cardiac internal excitation force value determination along the catheter 607 and detection of an abnormal excitation force 609 at a particular site. Processor 15 maps momentum value, mode value and deviation value to an indicator of heart chamber muscle condition and muscle and signal pathway condition using mapping information in repository 17.

The multi-channel signal momentum based cardiac status and function monitoring and analysis is applied in 2-dimension and 3-dimension heart mapping and is used in real time cardiac function diagnosis (determining and plotting EP signal momentum values over time). System 10 maps the multi-channel signal momentum information to abnormal tissue location, potential abnormal pathway identity and arrhythmia severity in a 2D or 3D visual representation of the heart to facilitate condition diagnosis by a clinician. System 10 advantageously identifies cardiac conditions without need for a stimulator and pacing pulse to be introduced into heart tissue and associated risk. System 10 advantageously analyzes excitation force distribution and clinical condition of a heart to expedite and facilitate heart condition diagnosis in emergency cardiac surgery cases.

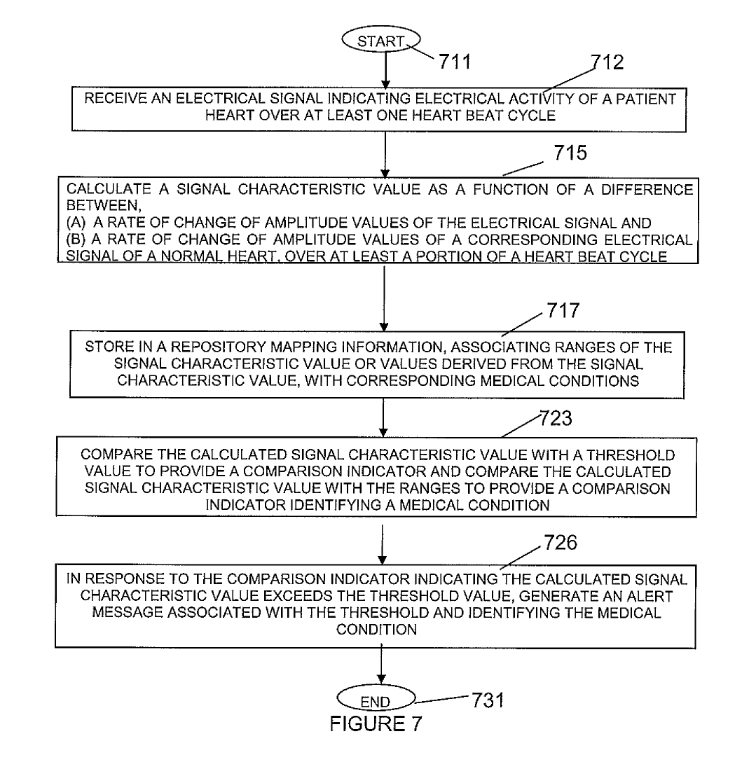

FIG. 7 shows a flowchart of a process used by system 10 for heart performance characterization and abnormality detection. In step 712 following the start at step 711, interface 12 receives an electrical signal and provides a digitized electrical signal indicating electrical activity of a patient heart over at least one heart beat cycle. In step 715, signal processor 15 calculates a signal characteristic value as a function of the digitized electrical signal comprising a difference between, (a) a rate of change of amplitude values of the electrical signal and (b) a rate of change of amplitude values of a corresponding electrical signal of a normal heart, over at least a portion of a heart beat cycle. In one embodiment, processor 15 calculates a signal characteristic value comprising a summation of values of rate of change of amplitude of the electrical signal over at least a portion of a heart beat cycle. The summation of values of rate of change of amplitude of the electrical signal over at least a portion of a heart beat cycle comprises an integral of rate of change of amplitude values of the electrical signal over at least a portion of a heart beat cycle.

Specifically, in one embodiment, the summation of values of rate of change of amplitude of the electrical signal over at least a portion of a heart beat cycle comprises,

$${\sum }_{t} m \cdot \frac{d A}{d t}$$

(m is the same for the same patient and may be set to 1).

In a further embodiment, the signal characteristic value is calculated as a function of a square of a difference between, (a) a rate of change of amplitude values of the electrical signal and (b) a rate of change of amplitude values of a corresponding electrical signal of a normal heart, over at least a portion of a heart beat cycle. The function in one embodiment comprises,

$${Momentum}_{AR\_mode} ( \% ) = \frac{{\sum }_{AR\_modeling} {( {a}_{1 i} - {a}_{2 i} )}^{2}}{{\sum }_{AR\_modeling} {( {a}_{1 i} )}^{2}}$$

where, a₁ᵢ and a₂ᵢ represent spectral (coefficients) of a Normal (healthy) and patient data series of rate of change of amplitude values.

Signal processor 15 calculates the signal characteristic value for a predetermined portion of a heart beat cycle (including an ST segment) in response to a heart (rate) synchronization signal. In one embodiment, processor 15 calculates the signal characteristic value as an averaged value over multiple heart beat cycles and in response to a heart rate synchronization signal. Processor 15 in step 717 stores, in repository 17, mapping information associating ranges of the signal characteristic value or values derived from the signal characteristic value, with corresponding medical conditions. The predetermined mapping information associates ranges of the signal characteristic value with particular patient demographic characteristics and with corresponding medical conditions. The system uses patient demographic data including at least one of, age, weight, gender and height in comparing the signal characteristic value or values derived from the signal characteristic value with the ranges and generating an alert message indicating a potential medical condition.

In step 723, comparator 20 compares the calculated signal characteristic value with a threshold value and with the ranges to provide a comparison indicator. The threshold value is derived from recorded electrical signal data for the patient or a population of patients. The population of patients has similar demographic characteristics including at least two of, (a) age, (b) weight, (c) gender and (d) height, to those of the patient. Signal processor 15 dynamically adjusts the threshold value in response to a determined sensitivity of arrhythmia detection. In response to the comparison indicator indicating the calculated signal characteristic value exceeds the threshold value or lies in a predetermined value range, patient monitor 19 in step 726 generates an alert message associated with the threshold and identifying the medical condition. The patient monitor substantially continuously monitors the comparison indicator for at least a 24 hour period. The process of FIG. 7 terminates at step 731.

A processor as used herein is a device for executing machine-readable instructions stored on a computer readable medium, for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a controller or microprocessor, for example, and is conditioned using executable instructions to perform special purpose functions not performed by a general purpose computer. A processor may be coupled (electrically and/or as comprising executable components) with any other processor enabling interaction and/or communication there-between. A user interface processor or generator is a known element comprising electronic circuitry or software or a combination of both for generating display images or portions thereof. A user interface comprises one or more display images enabling user interaction with a processor or other device.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A user interface (UI), as used herein, comprises one or more display images, generated by a user interface processor and enabling user interaction with a processor or other device and associated data acquisition and processing functions.

The UI also includes an executable procedure or executable application. The executable procedure or executable application conditions the user interface processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the UI display images in response to signals received from the input devices. In this way, the user interacts with the display image using the input devices, enabling user interaction with the processor or other device. The functions and process steps herein may be performed automatically or wholly or partially in response to user command. An activity (including a step) performed automatically is performed in response to executable instruction or device operation without user direct initiation of the activity.

The system and processes of FIGS. 1-7 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. The system maps calculated electrophysiological signal momentum values of different portions of cardiac signals to cardiac location and severity of cardiac pathology and improves differentiation and characterization of cardiac arrhythmias. Further, the processes and applications may, in alternative embodiments, be located on one or more (e.g., distributed) processing devices on a network linking the units of FIG. 1. Any of the functions and steps provided in FIGS. 1-7 may be implemented in hardware, software or a combination of both.

### APPENDIX

### AR Modeling and Analysis

An AR model may be regarded as a set of autocorrelation functions. AR modeling of a time series is based on an assumption that the most recent data points contain more information than the other data points, and that each value of the series can be predicted as a weighted sum of the previous values of the same series plus an error term. The AR model is defined by:

$$x [ n ] = {\sum }_{i = 1}^{M} {a}_{i} x [ n - i ] + ɛ [ n ]$$

where x[n] is a current value of the time series, a1 . . . aM are predictor (weighting) coefficients, M is the model order, indicating the number of the past values used to predict the current value, and ε[n] represents a one-step prediction error, i.e. the difference between the predicted value and the current value at this point. The AR model determines an analysis filter, through which the time series is filtered. This produces the prediction error sequence. In the model identification, the AR analysis filter uses the current and past input values to obtain the current output value. By using following equation:

$$ɛ [ n ] = x [ n ] - {\sum }_{i = 1}^{M} {a}_{i} x [ n - i ]$$

A filter with an impulse response [1, −a1, . . . , −aM], produces the prediction error sequence ε[n]. The predictor coefficients are estimated using least-squares minimization so that they produce the minimum error ε[n].

What is claimed is:

1. A system for heart performance characterization and abnormality detection, comprising:
an interface for receiving an electrical signal indicating electrical activity of a patient heart over at least one heart beat cycle;
a signal processor for calculating a signal characteristic value comprising a summation of values of rate of change of amplitude of said electrical signal over at least a portion of a heart beat cycle;
a comparator for comparing the calculated signal characteristic value with a threshold value to provide a comparison indicator; and
a patient monitor for in response to said comparison indicator indicating the calculated signal characteristic value exceeds the threshold value, generating an alert message associated with the threshold.

2. A system according to claim 1, wherein
said patient monitor, in response to said comparison indicator indicating the calculated signal characteristic value lies in a predetermined value range, generates an alert message associated with the value range.

3. A system according to claim 2, wherein
said patient monitor substantially continuously monitors said comparison indicator for at least a 24 hour period.

4. A system according to claim 1, wherein
said threshold value is derived from recorded electrical signal data for said patient.

5. A system according to claim 1, wherein
said threshold value is derived from recorded electrical signal data for a population of patients.

6. A system according to claim 5, wherein
said population of patients has similar demographic characteristics including at least two of (a) age, (b) weight, (c) gender and (d) height, to those of said patient.

7. A system according to claim 1, wherein
said signal processor dynamically adjusts said threshold value in response to a determined sensitivity of arrhythmia detection.

8. A system according to claim 1, wherein
said signal processor calculates said signal characteristic value for a predetermined portion of a heart beat cycle in response to a synchronization signal.

9. A system according to claim 8, wherein
said predetermined portion of said heart beat cycle includes an ST segment.

10. A system according to claim 1, wherein
said signal processor calculates said signal characteristic value as an averaged value over a plurality of heart beat cycles.

11. A system according to claim 1, wherein
said signal processor calculates said signal characteristic value in response to a heart rate synchronization signal.

12. A system according to claim 1, including
a repository of mapping information, associating ranges of the signal characteristic value or values derived from the signal characteristic value, with corresponding medical conditions and
said comparator compares the calculated signal characteristic value with said ranges to provide a comparison indicator identifying a medical condition and
said patient monitor generates an alert message identifying said medical condition.

13. A system according to claim 12, wherein
said predetermined mapping information associates ranges of the signal characteristic value with particular patient demographic characteristics and with corresponding medical conditions and said system uses patient demographic data including at least one of, age, weight, gender and height in comparing the signal characteristic value or values derived from the signal characteristic value with said ranges and generating an alert message indicating a potential medical condition.

14. A system according to claim 8, wherein
said interface provides a digitized electrical signal and
said signal processor calculates the signal characteristic value of the digitized electrical signal.

15. A system according to claim 1, wherein
said summation of values of rate of change of amplitude of said electrical signal over at least a portion of a heart beat cycle comprises an integral of rate of change of amplitude values of said electrical signal over at least a portion of a heart beat cycle.

16. A system according to claim 1, wherein
said summation of values of rate of change of amplitude of said electrical signal over at least a portion of a heart beat cycle comprises,
$${\sum }_{t} m \cdot \frac{d A}{d t}$$
(m is the same for the same patient and may be set to 1)

17. A system for heart performance characterization and abnormality detection, comprising:
an interface for receiving an electrical signal indicating electrical activity of a patient heart over at least one heart beat cycle;
a signal processor for calculating a signal characteristic value as a function of a difference between,
(a) a rate of change of amplitude values of said electrical signal and
(b) a rate of change of amplitude values of a corresponding electrical signal of a normal heart, over at least a portion of a heart beat cycle;
a comparator for comparing the calculated signal characteristic value with a threshold value to provide a comparison indicator; and
a patient monitor for in response to said comparison indicator indicating the calculated signal characteristic value exceeds the threshold value, generating an alert message associated with the threshold.

18. A system according to claim 17, wherein
said signal characteristic value is calculated as a function of a square of a difference between,
(a) a rate of change of amplitude values of said electrical signal and
(b) a rate of change of amplitude values of a corresponding electrical signal of a normal heart, over at least a portion of a heart beat cycle.

19. A system according to claim 18, wherein
said function comprises,
$${Momentum}_{AR\_mode} ( \% ) = \frac{{\sum }_{AR\_modeling} {( {a}_{1 i} - {a}_{2 i} )}^{2}}{{\sum }_{AR\_modeling} {( {a}_{1 i} )}^{2}}$$
where, a₁ᵢ and a₂ᵢ represent spectral (coefficients) of a Normal (healthy) and patient data series of rate of change of amplitude values.

20. A method for heart performance characterization and abnormality detection, comprising the activities of:
receiving an electrical signal indicating electrical activity of a patient heart over at least one heart beat cycle;
calculating a signal characteristic value comprising a summation of values of rate of change of amplitude of said electrical signal over at least a portion of a heart beat cycle;
comparing the calculated signal characteristic value with a threshold value to provide a comparison indicator; and
in response to said comparison indicator indicating the calculated signal characteristic value exceeds the threshold value, generating an alert message associated with the threshold.
